(19) United States
(12) Patent Application Publication
(10) Pub. No.: US 2016/0135709 A1
(43) Pub. Date: May 19, 2016

(54) METHOD AND DEVICE FOR A BIOELECTRIC IMPEDANCE ANALYSIS (BIA) OF THE BODY OF A PERSON

(71) Applicant: Tomczak Jörg, Eggstätt (DE)

(72) Inventor: Tomczak Jörg, Eggstätt (DE)

(21) Appl. No.: 14/392,263

(22) PCT Filed: Jun. 24, 2014
(86) PCT No.: PCT/DE2014/000318

§ 371 Date: Dec. 26, 2015

(30) Foreign Application Priority Data
Jun. 26, 2013 (DE) 10 2013 106 690.9

(51) Int. Cl. A61B 5/053; A61B 5/00

(52) U.S. Cl. CPC A61B 5/053; A61B 5/6841; A61B 5/7278; A61B 5/6823; A61B 5/6825; A61B 5/0015

(57) ABSTRACT

To perform a bioelectrical impedance measurement (BIA) of the body of a person several current pulse giving input electrodes and several voltage drop output electrodes need to be connected with the surface of the body. The voltage drop between the output electrodes of individual body sections is detected by the measurement. The measured values obtained are converted in combination with for example statistical data to a body structure and visceral risk analysis. In order for a BIA belly, BIA belly hand and BIA-hand-hand, when four hand electrodes and four abdominal electrodes are combined, a person can do a measurement in a simple manner, even without the often considerable effort of wiring the body, according to the invention the measuring template in the form of a bicycle handlebar (2). On its underside in the two middle template sectors (6, 7) it contains four abdomen electrodes and on its upper side (4) in the handles (8, 9) each contains two hand electrodes (12, 12′). The person holds it in a given arm position in a physiological simple manner with reproducible accuracy in a defined position and accordingly pressed against the abdomen.

The invention relates to a method and an apparatus for bioelectrical impedance measurement (BIA) of the body of a person, wherein a plurality of alternating current leading electrode pairs (here: input electrodes) and a plurality of the voltage drop measured sensitive electrode pairs (in this case output electrodes) have contact to the surface of the body and measure the bioelectrical impedance of the body segment between these output electrodes. The measured values together with stored, for example, statistical data, are converted to a body structure—and a visceral risk analysis.

During the measurement, a, in a measuring device generated alternating current in the kHz-area sent via the input electrodes into the body of the person generates an electromagnetic field, the three-dimensional diffuse between the input electrodes in all tissue layers and is measured using the output electrodes. In order to detect an as meaningful as possible body area during the measurement, the electrodes are placed on characteristic distinctive parts of the body and connected to a measuring device.

In order to determine a dimension in relation to body fat, a hand-foot measurement of the internal impedance of the body is already possible with only four electrodes. Here, input electrodes will be placed on the right foot and on the right hand of a person in the vicinity of output electrodes, so that during the measurement an electromagnetic field is built between the impulse giving electrodes of the right hand and the right foot. The measured potential difference between the output electrodes and the power of the passing current and the phase shift then represents the result of the impedance of the body.

In DE 602 05 976 T2 a gauge with eight electrodes is described used to evaluate or to assess the visceral body fat ratio. With the addition of a hand-hand and foot-foot bioelectrical impedance measurement and the body weight as well as blood pressure and pulse rate of the person can be measured. For this purpose, the measuring device is constructed in the form of an L-shaped scale, on whose base part four foot electrodes and on two side handles two hand electrodes are placed. To measure the person places the foot in a way that it touches the input electrodes with the toes and the output electrodes with the heels of the right and the left foot. At the same time both lateral handles are attached to the hands, whereby the contact with the input and output electrodes arranged there—hand electrodes is made.

To calculate the proportion of different body fat mass by determining the bio-electrical impedance of a body, the DE 11 2008 001 501 T5 proposed a foot-foot and hand-hand, combined with an abdominal measurement with a total of twelve electrodes. For the measurement a fastening element in the form of a belt with 4 electrodes—two inputs and two output electrodes on the abdomen so-as well via collars each with an input and output electrode fastened to the upper limbs, here at the wrists and each one input and output electrode on the lower limbs, here at the ankles to a person in lying position.

A disadvantage of known methods for measuring the bioelectrical impedance of a body is the often considerable expense of cabling, especially when involving a belly measurement, namely that a person cannot independently carry out such a measurement at least it is very difficult. In addition, it is required that this person must be without shoes and other legwear, such as support stockings also in order to be then measured in a lying position.

Starting from the prior described state of technology, the object of inventions is to provide a measuring device, with which a person can perform a BIA-belly, a BIA-abdominal hand and a BIA-hand hand measurement at the same time independently in a simple manner.

The stated task is procedurally solved with the characterizing features of claim 1 and the apparatus with the characterizing features of claim 5. The measuring template in the form of bicycle handlebars used self-sufficiently by a person in a simple manner performs BIA abdominal, a BIA-abdominal to hand and a BIA-hand to hand measurement simultaneously. The template is connected to a measuring module and has two input electrodes in the two middle template sections on the left and right, one output electrode each on the outer template sections on the left and right and an input electrode and an output electrode each on both handgrip on the outside of the template. To perform a measurement the person grips the measuring template on both handgrip with thumb and index finger and holds the template with the inner electrodes against the belly in a defined standing, sitting or lying position.

To produce the defined position the measuring template is ergonomically shaped in a way that a measurement can only be executed with predetermined arm position. Thus, the measurement can be carried out by different thick people, according to an advantageous development of the invention :the outer sections of the measuring template are elastic so that they can be adapted to the waist of the person during the measurement process.

The stencil center is marked with a label, such as a gap. To measure this marker is applied to the belly button so that the on the underside of the template arranged electrodes are touching the abdomen. This ensures that the abdominal electrodes are located laterally from the navel in the same position for each measurement. Advantageously, the electrodes are wetted with a drop of electrode gel.

The abdomen electrodes are arranged symmetrically around the mark, so that for example the output electrodes are 7 cm away from the mark to the left and right and the input electrodes are 12 cm away from the mark.

Advantageously, compared to other known measuring devices for the measurement often complicated fastening units and cabling for the electrodes are not required.

The readings produced by, for example 50 kHz alternating current pulses at a measurement frequency are detected by the measurement module and can be used with a computer connected unit or calculation unit for creating a body structure and visceral risk analysis. But according to the invention it is also possible to perform the measurement with a further and/or several measuring frequencies between 1-1000 kHz AC pulses.

The measurement module that includes a current Impulser producers for the input electrodes and a voltage drop meter for the output electrodes to perform the measurement is inventively incorporated as an integral part in the measurement template. Alternatively, the measuring module can be constructed as a separate unit and connected to the measuring template over a corresponding device.

The measurement module can for example be inserted in a contact pin header arranged at the top of the measuring template, which open the connecting lines of the electrodes, or the measuring module is connected via electrode lead wires containing line with the measuring template or the integrated measuring module transmits the measured data per radio to an external evaluation unit.

The measurement carried out using the measurement template are used to perform a visceral risk analysis. The BIA belly states: the larger the measured resistance (irrelevant whether resistance or impedance) is the greater visceral risk. Approximately 50% of the resistance thereby is generated by the subcutaneous fatty tissue. The second half is shared between the other components of the abdominal cavity, such as liquids (intra- and extracellular), abdominal muscles, intramuscular fat accumulation, abdominal organs, visceral fat accumulation, bone respectively spine. In summary, the BIA belly measurement is a high quality statement for the thickness of the subcutaneous fat layer and beyond gives a statement about other internal belly compartments.

The BIA abdominal to hand and BIA-hand to hand is also not a pure fat measurement, but a body structure analysis, particularly for the relation of the muscle/fat ratio. By combining BIA belly, BIA abdominal to hand and BIA hand to hand measurements existing uncertainties regarding the visceral risk analysis can be largely reduced and expanded with a body structure analysis.

The inventive method uses four power-giving input electrodes (both index fingers and the two outer abdominal electrodes) on the measuring template, which can be combined to six possible pair of connections. Similarly, there are four output electrodes on the measuring template which also can be combined in six possible pair connections. Altogether 36 measuring sections are possible, which can be measured in theory one after another. Realistically, however, only some of these measurement paths are necessary for meaningful calculation.

Further details and advantages of the invention will be explained with reference to schematic drawings shown in execution examples.

Shown are:

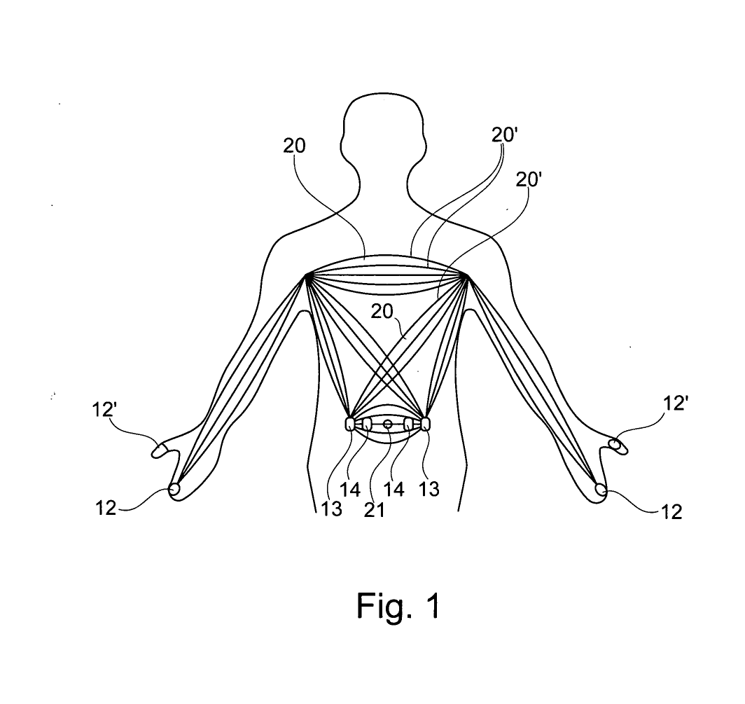

FIG. 1 possible connections and electromagnetic field lines between four input electrodes,

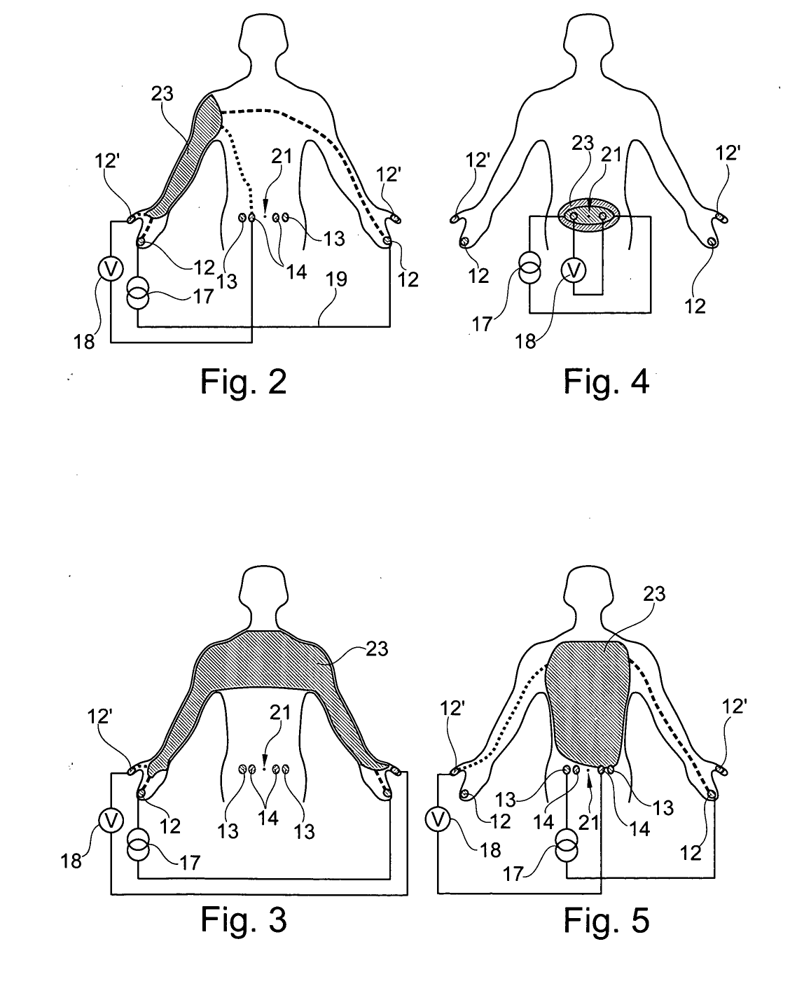

FIG. 2-5 Measuring arrangements on different areas of the body,

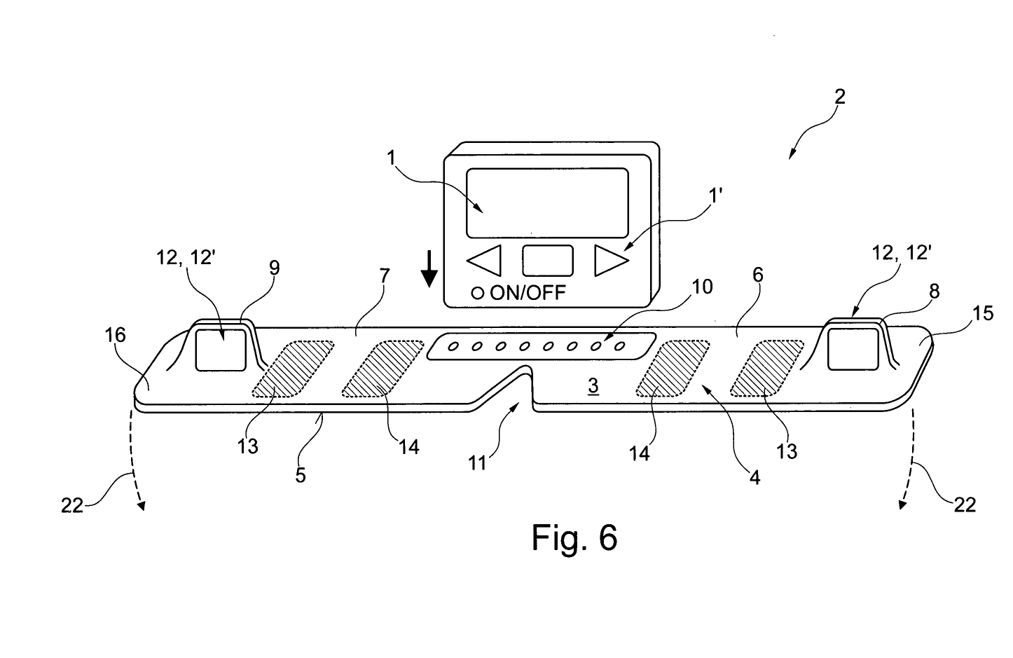

FIG. 6 a measuring template of the invention in a perspective front view.

FIG. 1 represents a front view of the torso of a person. In both hands in the area of the index finger an input electrode is located 12 as well as an output electrode in the area of the thumb 12′.

Furthermore, located to the right and left of the navel 21 each output electrode 14 and next further outwardly right and left from the navel 21 an input electrode. 13

The resulting field lines 20 of a corresponding activation between through electrodes connecting lines connected (19, FIG. 2) with an intermediate current pulse generator (17, FIG. 2-5) to the input electrodes 12 and 13′, respectively the generated electromagnetic fields 20 are correspondingly located in the upper body of the person.

These electromagnetic fields 20 and respectively their mitigation are measured through electrodes connecting lines (19, FIG. 2) with an intermediate voltage drop diameter (18, FIG. 2-5) interconnected output electrodes 12′ and 14th

In the schematic diagram of FIG. 1 it can be clearly seen that a metrological relevant field throughout upper body, abdomen and arms of a person can be generated with only four input electrodes. By selectively combining individual electrode pairs very specific individual measuring ranges 23 of the body can be captured metrologically, as is shown schematically in the following drawings 2 to 5.

In the FIG. 2 a measuring range is drawn dimly over the right arm 23, which is to be determined by measurement. In order to realize the measurement, the input electrodes 12 of the left and right index finger are activated. The output electrodes 12′ of the right thumb of the right output electrode 14 of the abdominal electrode measure the voltage drop and the phase shift of this field.

In the FIG. 3 the output electrodes are changed while having identical input electrodes 12 of both index-finger compared to of FIG. 2. The output electrode 12′ of the left and right thumb determine the measuring range, the in FIG. 3 dark highlighted measuring range 23 of the shoulder, upper chest and both arms (BIA-hand-hand). The abdominal electrodes 13, 14 were not used for the measurement in this example.

FIG. 4 shows an exclusive measurement of the lower abdomen is (BIA belly) according to the dark highlighted range of measurement 23. For this purpose, only the abdominal electrodes 13, 14 used in this measurement whereas the two outer input electrodes 13, 14 generate the field while the two inner output electrodes measure the voltage drop and the phase shift. In this example, the hand electrodes are not included in the measurement.

A measurement of the entire upper body (BIA belly-hand), shown in FIG. 5 as a dark highlighted measurement range is shown 23. The following electrode combination is possible: As input electrodes act 12 of the left index and 13 of the right abdominal electrode. As output electrodes act 12′ of the right thumb, and the right of the abdominal electrode 14.

In particular, the electrode combinations of FIGS. 2 and 5, are used simultaneously in the abdominal electrodes and hand electrodes to perform a measurement with the invented measuring template. Those complement each other in the quality of the statement about medically relevant “visceral risk”.

FIG. 6 is a perspective front view of the schematically construction of the measuring template 2 as shown as a sketch. The measuring template in the form of a bicycle handlebar 2 consists of an ergonomically designed belt-shaped body 3. It contains handgrip 8, 9 on the outer region on the left and right side of the template 4 in the region of 15, 16. Due to these handgrip 8, 9, and the ergonomical form of the measuring template 2 it can only be used in a predetermined arm position and placed on-the own belly. To support a reproducible holding position on the belly, the outer template sections 15, 16 is elastically so that they bend in the direction of arrow 22 and can adjust the waist while keeping the measuring template 2.

In each of the handles 8, 9 is an input electrode 12 and an output electrode 12′. They are arranged one behind the other in a way on the measuring template, that they inevitably 2 come currencies—end with thumb and index finger grip in contact (In the FIG. 6 only the output electrode 12′ is visible).

The bottom side 5 of the measuring template 2 contains one input 13 electrode and one output electrode 14 each on the right and left side is in the middle part of the template 6, 7. The output electrodes 14 are positioned 7 cm to the left and right and the input electrodes 13 11 cm to the left and to the right from the marked middle of the template 11 (in the FIG. 6, the electrode pairs 13 and 14 on the bottom are shown as hatched Fields with dashed edges, since they would normally not be visible from this angle).

The mark 11 of the center of the measuring template serves as an aid to position it at the belly button (21, FIG. 1) before starting the measurement.

In the illustrated embodiment no electrode connection cables are drawn, as they are integrated in the measuring template and open into a pin bar 10 on the upper side 4 of the measuring template 2.

The current pulse generator which is necessary for the measurement and chip voltage drop measure are also not shown, since they are components of the measurement module 1 with display and control keys 1, which is designed in a way that it can be inserted into the pin header 10.

### LIST OF REFERENCE NUMBERS

1 measuring module with display

1 operation buttons

2 measuring template

3 band-shaped main body of the measuring template

4 top of the base body

5 underside of the base body

6, 7 middle part of the measuring template

8, 9 handgrip of the measuring template

10 pin bar

11 mark

12 input electrodes for the index finger

12 output electrodes for the thumb

13 input electrode in the middle part of the measuring template

14 output electrode in the middle part of the measuring template

15, 16 outer template sections

17 current pulse generator

18 voltage drop measure

19 electrode connection cables

20 electromagnetic fields

20 electromagnetic field lines

21 belly button

22 arrow of the possible bending

23 measuring range

1. Method for the bioelectrical impedance measurement (BIA) of the body of a person, wherein a plurality of a current pulse giving input electrodes and a plurality of the voltage drop measured sensitive output electrodes with the surface of the body brought into contact and between these electrodes the bioelectrical impedance of the body metrological recorded and the measured values obtained with stored, for example, statistical data converted to a body structure—and visceral risk analysis, characterized in that via a constructed like the form of bicycle handlebars, with a measurement module (1) connected measuring template (2), which in the middle stencil portions (6, 7) respectively to the left and right have an input electrode (13) and an output electrode (14) and at the handles (8, 9) of the outer stencil portions (15, 16) respectively left and right, an input electrode (12) and an output electrode (12′), is performed by the person self-sufficient in a simple manner with reproducible accuracy simultaneously a BIA-belly, a BIA abdominal hand and a BIA-hand-hand, wherefore the person holds the measurement template (2) at the handles (8, 9)—with thumb and index finger contact to the local electrodes (12, 12′) and their middle part of the template (6, 7) with the electrodes (13, 14) in a defined position held against the stomach.

2. The method of claim 1, characterized in a way to secure the defined position during the measurement the measuring template (2) having a predetermined arm position and is aligned with its center at the navel (21).

3. The method of claim 1 or 2, characterized in a way that the measurement is carried out with a frequency of 50 kHz AC pulses and without additional mounting or wiring on the body and the measurements obtained with measuring module (1) with an integrated computer or calculating unit are used to generate a body structure and visceral risk analysis.

4. The method according to claim 3, characterized in a way that the measurement be carried out with a further and/or several measuring frequencies between 1-1000 kHz AC pulses.

5. A device for performing the method according to one or more of claims 1 to 4, characterized by:
one with a measurement module (1) connected, measuring template in the shape of a bike handlebar (2), which's band-shaped base body (3) is ergonomically shaped in a way that the measuring template (2) with it's at the outer template sections (15, 16) arranged handgrip (8, 9) can be held in only a predetermined arm position suitable to the stomach and pressed against it;
in the electrodes (12, 12′) arranged in the handgrip (8, 9), while holding the measuring template (2′) each thumb inevitably gets in contact to the output electrode (12′) and each index finger to the input electrode (12);
on the underside (5) of the measuring template (2) in the middle part of the template (6, 7) left and right from the middle part of the template arranged input and output electrodes (13, 14).

6. The method of claim 5, characterized in a way that the right positioning of the templates center on the navel (21), is marked with for example an opening on the template center (11).

7. The method according to claim 5 or 6, characterized in a way that the abdominal electrodes (13, 14) on the underside (5) of the measurement template (2) are arranged symmetrical around the marking (11), that the output electrodes (14) for example, left and right 7 cm and further out to the input electrodes are arranged for example the left and right 12 cm away from the mark (11).

8. The method according claim 5, 6 or 7, characterized in a way that the outer template sections (15, 16) of the belt-shaped base body (3) are elastic enough to adapt to the abdominal girth during the measurement process while holding the measuring template (2),

9. The method according to claim 5, 6, 7 or 8, characterized in a way that the to the measurement template (2) connected measuring module (1) has a current pulse generator (17) for the input electrodes (12, 13) and a voltage drop meter (18) for the output electrodes (14, 12′), and is equipped with an integrated computer unit or calculation unit for creating a body structure—and visceral risk analysis.

10. The method according to claim 9, characterized in a way that the measuring module (1) and the connection line electrode are integrated as an integral part in the measurement template (2).

11. The method according to claim 9, characterized in a way that the measuring module (1) is inserted in one on the upper side (4) of the measurement template (2) arranged contact pin strip (10) into which the connecting line (19) of the electrodes discharge.

12. The method according to claim 9, characterized in a way that the measuring module is connected (1) via an electrode connecting line (19) in a containing common line with the measuring template (2).

13. The method according to claim 10, 11 or 12, characterized in a way that the measuring module (1) transmits the measurement data with an integrated radio module to an external station.
